(19) United States
(12) Patent Application Publication
(10) Pub. No.: US 2014/0274985 A1
(43) Pub. Date: Sep. 18, 2014

(54) SYSTEMIC ADMINISTRATION OF ANDROGEN IN TREATING DRY EYE SYNDROME

(71) Applicants: Raman Malhotra, Oxted (GB); Matthew Long, Horley (GB)

(72) Inventors: Raman Malhotra, Oxted (GB); Matthew Long, Horley (GB)

(21) Appl. No.: 14/201,063

(22) Filed: Mar. 7, 2014

Related U.S. Application Data
(60) Provisional application No. 61/789,066, filed on Mar. 15, 2013.

(51) Int. Cl. A61K 31/568

(52) U.S. Cl. CPC A61K 31/568
USPC 514/179

(57) ABSTRACT

The present disclosure provides a method for treating dry eye syndrome that comprises systemically administering to a patient in need thereof an effective amount of an androgen, including transdermal or subcutaneous delivery of the androgen.

### BACKGROUND

1. Technical Field

This disclosure relates to treating dry eye syndrome via systemic administration of an androgen.

2. Description of the Related Art

Dry eye syndrome is one of the most common disorders of the eye. It is caused by a lack of adequate tears or poor quality of tears (i.e., an imbalance in the composition of the tears). Patients with dry eye syndrome may have pain, light sensitivity, a gritty sensation in the eye, a feeling of a foreign body or sand in the eye, eye itching, eye redness, stringy mucus in or around the eye, increased eye irritation from smoke or wind, eye fatigue, difficulties wearing contact lenses, period of excessive tearing, or blurring of vision. Potential complications of dry eyes include more frequent eye infections, scarring on the surface of the eye, and decreased quality of life.

Current treatments of dry eyes include artificial tear drops and ointments, temporary punctal occlusion, permanent punctal occlusion, surgery to permanently close the ducts that drain tears into the nose to allow more tears to remain around the eye, antibiotics to reduce eyelid inflammation, and covering the eye with a specific contact lens to trap moisture close to the eye. Transdermal delivery of androgenic hormones locally to the ocular surface, immediate vicinity of an eye, or the adnexa of the eye (i.e., the tissue adjacent to and surrounding the eyeball) was also reported for treating dry eye syndrome. However, such treatments require multiple daily topical applications of androgen and may cause skin sensitivity and irritation at application sites.

### SUMMARY

The present disclosure provides a method for treating dry eye syndrome, including aqueous tear-deficient dry eye syndrome and especially evaporative dry eye syndrome. The method comprises systemically administering to a patient in need thereof an effective amount of an androgen. The systemic administration of an androgen may be by transdermal delivery of an androgen (e.g., via an androgen patch or gel) or subcutaneous delivery of an androgen (e.g., via an androgen depot or implant). The androgen may be administered at a dose range of 100-1300 μg per day. In certain embodiments, the androgen is testosterone. The patient to be treated may have a low serum androgen level.

In the following description, any ranges provided herein include all the values in the ranges. It should also be noted that the term “or” is generally employed in its sense including “and/or” (i.e., to mean either one, both, or any combination thereof of the alternatives) unless the content clearly dictates otherwise. Also, as used in this specification and the appended claims, the singular forms “a,” “an,” and “the” include plural referents unless the content clearly dictates otherwise.

### BRIEF DESCRIPTION OF THE DRAWINGS

FIG. 1 is a graph showing comparison of Ocular Surface Disease Index (OSDI) scores at baseline and during 3 weeks androgen patch on.

FIG. 2 is a graph showing comparison of Ocular Surface Disease Index (OSDI) scores after 3 weeks androgen patch on and 3 weeks patch off.

### DETAILED DESCRIPTION

The present disclosure provides a method for treating dry eye syndrome that comprises systemic administration to a patient of dry eye syndrome an effective amount of androgen. The method is based on the discovery that systemic administration of androgen is effective in treating dry eye syndrome, including aqueous tear-deficient dry eye syndrome and especially evaporative dry eye syndrome. This discovery is unexpected because a drug may have different efficacies or adverse effects when delivered in different manners (e.g., locally v. systemically), and because a drug effective for an indication when delivered locally may be ineffective or toxic for the same indication when delivered systemically (see, Izazola-Conde et al., Proc. West. Pharmacol. Soc. 54:68-71, 2011).

The improvement in dry eye by the method disclosed herein is potentially more sustainable than local administration of androgen to the tissue adjacent to and surrounding the eyeball. In addition, the method provides additional benefits to patients suffering from dry eye syndrome due to a low serum androgen level. Such additional benefits include increasing sex drive, reducing depression, increasing a sense of well-being, improving concentration, increasing muscle mass, reducing body fat, improving cholesterol levels, increasing hemoglobin, and reducing osteoporosis.

“Dry eye syndrome” refers to a condition when tears of a subject are not able to provide adequate moisture for the eyes resulting from a lack of adequate tears or an imbalance in the composition of the tears.

Tears are a film comprised of three layers: the outermost lipid layer, the middle aqueous layer, and the innermost mucous layer. The lipid layer is secreted by the meibomain gland and reduces evaporation, the aqueous layer supplies oxygen and a mixture of electrolytes to surface eye cells, and the innermost mucous layer is produced by the conjunctiva goblet cells that attaches the tear film to the corneal surface. Each of these layers is critical to producing and maintaining tear film that hydrates, nourishes and protects the ocular surface from infection.

Dry eye syndrome may be classified into two categories: aqueous tear-deficient dry eye (ATDDE) and evaporative dry eye (EDE) (see, Ocul Surf 5(2):75-92, 2007). In ATDDE, lacrimal tear secretion is reduced, either through glandular disease or destruction. In contrast, EDE may occur in the presence of normal tear gland function and is most frequently due to increased evaporation from the ocular surface secondary to a deficient outmost lipid layer in the tear film (see, Ocul Surf 5(2):75-92, 2007; Baum, Ophthalmology 117(7):1285-6, 2010). The most common cause of EDE is meibomian gland dysfunction or a decrease in secretion of meibum.

Symptoms of dry eye syndrome include stinging, burning, scratchy, or gritty sensation, light sensitivity, and/or a feeling of a foreign body or sand in the eye, eye itching, eye redness, stringy mucus in or around the eye, increased eye irritation from smoke or wind, eye fatigue, difficulties wearing contact lenses, period of excessive tearing, or blurring of vision.

Various conditions cause dry eyes. In addition to a deficiency in the tear-flow system of the eye, other conditions that may cause dry eyes include the natural aging process, especially menopause; side effects of certain drugs such as antihistamines and birth control pills; diseases that affect the ability to make tears, such as Sjogren's syndrome, rheumatoid arthritis, and collagen vascular diseases; and structural problems with the eye lids that don't allow them to close properly.

Tests for diagnosing dry eye syndrome and determining its causes are known in the art, including a comprehensive eye exam, measuring the volume of tears, and determining the quality of tears. Exemplary tests for screening patients for dye eye syndrome, especially for evaporative dry eye syndrome, include the ocular surface disease index questionnaire, tear film break-up-time, a slit-lamp ocular surface examination, Schirmer's test with anesthetic, meibography (an in-vivo means to assess the structure of the meibomian gland), and other tests described in the Example provided herein.

As understood by a person skilled in the medical art, the terms, “treat” and “treatment,” refer to medical management of a disease, disorder, or condition of a subject (i.e., patient) (see, e.g., Stedman's Medical Dictionary). “Treating dry eye syndrome” refers to reducing the number of symptoms or decreasing the severity of one or more symptoms of dry eye syndrome.

The effectiveness of a treatment of dry eye syndrome can readily be determined by a person skilled in the medical art using one or a combination of diagnostic methods and comparing symptoms of patients that have received the treatment with those of patients without such a treatment or with placebo treatment. Alternatively, symptoms of one or more patients after a treatment may be compared to those of the same patient(s) before the treatment.

Patients that may be treated by the method provided herein include humans (both men and women) and non-human mammals, including dogs, cats, pigs, sheep, and cattle. This method is particularly useful for treating patients suffering from evaporative dry eye syndrome. The patients to be treated may have low serum androgen levels, such as women with lower than 0.2 (e.g., lower than 0.3, 0.4, or 0.5) nmol/L serum level of total testosterone, and men with lower than 3 (e.g., lower than 4, 5, 6, 7, 8, 9, 10, 11, 12, 13, 14, or 15) nmol/L serum level of total testosterone. In certain embodiments, the patient to be treated is a post-menopausal woman.

“Androgen” refers to any natural or synthetic compound that stimulates or controls the development and maintenance of male characteristics in vertebrates by binding to androgen receptors, and precursors, metabolites, isomers, analogues, esters, salts, and derivatives (e.g., phosphorylated ester derivatives) of such a compound. The primary and most well-known androgen is testosterone. Other androgens include dehydroepiandrosterone (DHEA), androstenedione, androstenediol, androserone, dihydrotestosterone (DHT), methandrostenoine, oxymetholone, ethylestrenol, oxandrolone, nandrolone phenpropionate, nandrolone decandate, stanozolol and dromostanolone propionate. Additional exemplary androgens are described in U.S. Application Publication Nos. 2006/0211660 and 2012/0190661).

Natural androgens are produced by leydig cells in men and influence them physically, emotionally and sexually. Androgens are also produced in the adrenal gland and the ovary in women and may contribute to maintaining normal ovarian function, bone metabolism, cognition, and sexual behavior in women.

An androgen may be present in a pharmaceutical composition when administered to a patient suffering from dry eye syndrome. The pharmaceutical composition may contain, in addition to the androgen, a pharmaceutically acceptable carrier or excipient. Pharmaceutical acceptable carriers or excipients are well known in the pharmaceutical art and described, for example, in Rowe et al., Handbook of Pharmaceutical Excipients: A Comprehensive Guide to Uses, Properties, and Safety, 5^{th} Ed., 2006, and in Remington: The Science and Practice of Pharmacy (Gennaro, 21^{st} Ed. Mack Pub. Co., Easton, Pa. (2005)).

Exemplary pharmaceutically acceptable carriers include water; organic solvents such as alcohols (particularly lower alcohols), glycols (such as glycerin), aliphatic alcohols (such as alonolin); mixtures of water and organic solvents (such as water and alcohol), and mixtures of organic solvents such as alcohol and glycerin (optionally also with water); lipid-based materials such as fatty acids, acylglycerols (including oils, such as mineral oil, and fats of natural or synthetic origin), phospholipid (including phosphoglycerides and lecithin), sphingolipids and waxes, protein-based materials such as collagen and gelatin; silicon-based materials (both non-volatile and volatile) such as cyclomethicone, demethiconol and dimethicone copolyol (Dow Corning); hydrocarbon-based materials such as petrolatum and squalane; anionic, cationic and amphoteric surfactants; sustained-release vehicles such as microsponges and polymer matrices; stabilizing and suspending agents; emulsifying agents, and other vehicles and vehicle components suitable for administration to the skin, as well as mixtures of topical vehicle components as identified above or otherwise known to the art. The composition may further include components adapted to improve the stability or effectiveness of the applied formulation, such as preservatives, antioxidants, skin penetration enhancers (e.g., triglycerides and phospholipids such as phosphatidylcholine, phosphatidylethanolamine, and phosphatidylserine), sustained release materials, neutralizing agents, tonicity agents, buffering agents, thickener, and the like. Additional examples of pharmaceutical carriers, excipients and other optional components that may be present in pharmaceutical compositions that comprise an androgen may be found in U.S. Pat. Nos. 5,869,090 and 6,659,985, U.S. Application Publication Nos. 2012/0190661, 2012/0164213, and 2006/021660.

In certain embodiments, the pharmaceutical composition that comprises an androgen is capable of releasing the androgen in a sustained manner so that the level of androgen available to the patient is maintained at a desirable level over a desired period of time. For example, the desirable level of total serum testosterone may be in the range of about 100 to 1300 ng/dL. The desired period of time may be at least 6 hours, such as at least 8, 10, 12, 24 hours, 2, 3, 4, 5, 6, 7 days, or 2, 3, or 4 weeks. Methods for preparing sustained release compositions of androgen are known in the art, such as described in U.S. Application Publication No. 2004/0127476.

Androgen-containing pharmaceutical compositions may be in a form of patch, gel, cream, depot, or implant.

Exemplary androgen patches include those described in U.S. Pat. No. 5,869,090 and INTRINSA®. INTRINSA® is a testosterone patch by Procter & Gamble designed to treat female sexual dysfunction. The recommended daily dose of testosterone is 300 micrograms. This is achieved by applying the patch twice weekly on a continuous basis. Because androgen patches has a reservoir of the active androgen substance to be delivered over a period of time, they may be considered as a type of depot preparation.

Examples of useful androgen gel are TESTOGEL® (Bayer, Australia) and ANDROGEL® (United Pharmaceuticals, Deerfield Ill.). TESTOGEL® contains 50 mg testosterone in 5 g gel and is prescribed for replacing the body's natural testosterone when no enough is made by the body. ANDROGEL® 1% is a clear colorless hydroalcoholic gel containing 1% testosterone and provides continuous transdermal delivery of testosterone for 24 hours following a single application to the skin. Its inactive ingredients include carbomer 980, ethanol, isopropyl myristate, purified water, and sodium hydroxide.

Exemplary androgen creams include those described in U.S. Pat. No. 6,659,985 and in Allen, “Testosterone propionate” US Pharmacist, April 1992, pp. 68-72).

Exemplary androgen depots include PRIMOTESTON® Depot (Bayer, Australia). 1 ml PRIMOTESTON® Depot contains 250 mg testosterone enathate (equivalent to about 180 mg testosterone). Its inactive ingredients include benzyl benzoate and castor oil.

Exemplary androgen implants include testosterone implants by Schering-Plough Pty Ltd., Australia. They are for subcutaneous use, and contain 100 mg or 200 mg fused crystalline testosterone without any excipients. The estimated rate of release of testosterone is 0.65 mg per pay per 100 mg implant and 1.2-1.3 mg per day per 200 mg implant. Testosterone implants last from 6 to 9 months.

The method of treating dry eye syndrome disclosed herein comprises systemic administration of an androgen. “Systemic administration,” as used herein, refers to administration of an androgen other than local administration to the eye or a tissue, immediate vicinity of an eye, or the adnexa of the eye (i.e., the tissue adjacent to and surrounding the eyeball). After systemic administration, the androgen reaches the eye via bloodstream. Systemic administration may take place via enteral administration (absorption of an androgen through the gastrointestinal tract, such as oral administration) or parenteral administration (generally by injection, infusion, or implantation).

Systemic administration of an androgen may be also via transdermal delivery of the androgen by topically applying an androgen or androgen-containing pharmaceutical composition to the skin of a patient at a location distanced from the eye, such as at the arm, shoulder, abdomen, and buttock. In certain embodiments, the skin to which the androgen or androgen-containing pharmaceutical composition is applied is not facial skin. The androgen-containing pharmaceutical composition for topical application may be in a form of an androgen patch, gel or cream.

Systemic administration of an androgen may also be via subcutaneous delivery of the androgen by subcutaneous injection of androgen depots or subcutaneous insertion of androgen implants, preferably into an area where there is relatively little movement or blood supply, such as the lower abdominal wall or the buttock.

A “therapeutically effective amount” of an androgen refers to the amount of the androgen sufficient to result in reducing the number or severity of symptoms of dry eye syndrome in a statistically significant manner. Such an amount may be determined or adjusted depending on various factors include the specific androgen, the route of administration, the patient's condition such as the severity of symptoms, general health status, as well as age, gender, and other factors apparent to a person skilled in the medical art.

In certain embodiments, an androgen, such as testosterone, is released at a dose range of 100-1300 μg/day, such as 100-300, 300-500, 500-800, 800-1000, and 1000-1300 μg/day.

In some embodiments, an androgen, such as testosterone, is released in the amount so that the serum level is in the normal range of healthy women or healthy men. For example, testosterone may be administered a female human patient so that her serum level of total testosterone is at least 0.2 nmol/L, such as at least 0.3, 0.4, 0.5, 0.6, 0.7, 0.8, 0.9, 1.0, 2.0, 3.0, 4.0, or 5.0 nmol/L. Testosterone may also be administered to a male human patient so that his serum level of total testosterone is at least 2.0 nmol/L, such as at least 3.0, 4.0, 5.0, 6.0, 7.0, 8.0, 9.0, 10.0, 15.0, 20.0, 25.0 or 30.0 nmol/L.

Also contemplated is the administration of an androgen in combination with a second agent useful in treating dry eye syndrome. Exemplary second agents include estrogen and analogs thereof, progesterone, antibiotics to reduce eyelid inflammation, and cyclosporine or corticosteroids to control cornea inflammation.

The second agent may be administered to a patient via the same route as an androgen (e.g., transdermally or subcutaneously). Alternatively, the second agent may be administered to a patient via a route different from androgen administration route.

In certain embodiments, an androgen and a second agent useful in treating dry eye syndrome act synergistically to generate a combined effect greater than the sum of the individual effects of androgen and the second agent when administered alone. In certain other embodiments, an androgen and a second agent useful in treating dry eye syndrome act additively to generate a combined effect the same as or similar to the sum of the individual effects of each agent when administered alone.

It is contemplated the androgen and the second agent may be given simultaneously in the same formulation. Alternatively, the second agents may be administered in a separate formulation but concurrently (i.e., given within less than one hour of each other).

In certain embodiments, the second agent useful in treating dry eye syndrome may be administered prior to administration of an androgen (e.g., at least one hour before the start of the androgen treatment). It is also contemplated that the second agent may be administered subsequent to administration of the therapeutic agent (e.g., at least one hour after the start of the androgen treatment).

The following example is for illustration and is not limiting.

### EXAMPLE

This example evaluates the effect of transdermal androgen patch therapy as an adjunct to conventional therapy in patients with evaporative dry eye (EDE) and low androgen levels. The results show that in patients with androgen deficiency, transdermal androgen patch therapy appears to provide a subjective and objective improvement of EDE as an adjunct to conventional therapy.

### Methods

This is a retrospective review of case notes in the private practice of the present inventors. Patients with persistent and symptomatic EDE syndrome despite topical therapy including ocular lubricants, dietary flaxseed oil supplements, daily warm compresses, reduction of dietary dehydrating factors, use of humidifiers where convenient, etc. were screened for low serum testosterone levels, or at the lower border of the normal range were referred to a gynecologist for consideration of transdermal androgen patch therapy.

All clinic patients for EDE syndrome were screened. Patients were identified as having EDE by the presence of dry-eye symptoms, early tear film break-up-time (TFBUT), a normal Schirmer's test at 5 minutes with anesthetic, eyelid margin abnormalities including irregularity or thickening of the eyelid margin, vascular engorgement, plugging of the meibomian orifices, an indistinct muco-cutaneous junction and a thick and reduced meibomian gland secretion and no significant lagophthalmos on blink (blink lagophthalmos). Patients with anatomical lid anomalies (intrinsic cause of EDE), drug toxicity & contact lens (extrinsic cause of EDE) were excluded. Baseline blood tests included full blood count, urea, electrolytes and creatinine, serum testosterone, sex hormone binding globulin (SHBG), oestradiol, and progesterone. In systemic circulation, testosterone, binds to sex hormone-binding globulin (SHBG) and albumin, leaving behind only 1-2% of the hormone unbound in the circulation. Serum antinuclear antibodies, anti-ro, anti-la antibodies and rheumatoid factor were also requested if Schirmer's tests were reduced (5 or less) and/or signs of aqueous deficiency were present and/or systemic symptoms were evident. For all patients referred to a gynecologist, a detailed history of symptoms including the Ocular Surface Disease Index (OSDI) questionnaire (a 12-item questionnaire for assessing the symptoms of ocular irritation consistent with dry eye disease and their impact on vision-related functioning) developed by the Outcomes Research Group (Allergan Inc, Irvine, Calif., USA) were documented (Schiffman et al., Arch Ophthalmol 118(5):615-21, 2000). Mean total OSDI scores were calculated at baseline (pre-patching), after 3 weeks of patching and a subsequent cycle of 3 weeks patch on and 3 weeks patch off.

At baseline and follow-up visits, best-corrected Snellen distance visual acuity (BCVA) was assessed. A detailed slit lamp examination was performed to assess eyelid margin position and inflammation, meibomian gland dysfunction, punctal position and patency, blink lagophthalmos, conjunctival injection and tear film break-up-time (TFBUT) after instillation of one drop of 2% Fluorescein dye (Minims, Bausch & Lomb, USA) on to the bulbar conjunctiva. The patient was instructed to blink naturally, without squeezing, several times to distribute the fluorescein. Within 10-30 seconds of the fluorescein instillation, the patient was asked to stare straight ahead without blinking. The cornea was viewed on slit lamp with cobalt blue filter. TFBUT was the time between last complete blink and first appearance of growing micelle. The Schirmer's test was performed after instilling one drop of topical anesthetic, Oxybuprocaine Minims (Bausch & Lomb, USA) in the inferior conjunctival formix and the Schirmer paper strip (SNO strips, Chauvin Pharmaceuticals Ltd, Kingston Upon Thames, UK) were inserted over the lower lid margin, midway between the middle and outer third. Patient was instructed to blink normally and the wetting on the paper was measured after 5 minutes with a measuring scale. In patients with co-existing symptoms of epiphora, additional assessment of the fluorescein dye retention test (FDRT) and syringing were performed. A gynecological history, including symptoms attributable to low serum testosterone, was documented. Patients were counseled on the possible benefits, risks and the off-label nature of androgen patch therapy for this indication. In addition, the published literature regarding Intrinsa® transdermal androgen patch therapy (available on: http://www.medicines.org.uk/EMC/medicine/19592/SPC/Intrinsa®+300+micrograms+24+hours+transdermal+patch/#furtherinfo) was provided to each patient and they were encouraged to discuss this further with their general practitioner (GP).

Patients who then requested treatment were commenced on Intrinsa® therapy. The INTRINSA® patch is 28 cm² and worn on the lower abdominal skin below the waist (see, Testosterone patches for female sexual dysfunction. Drug Ther Bull 47(3):30-4, 2009).

Patients were instructed to apply the patch on the lower abdomen twice weekly for three weeks. The patient's GP was requested to repeat serum testosterone and serum SHBG levels after three weeks. The patch was discontinued for a further 3 weeks interval and the patient's GP was requested to re-check serum testosterone and SHBG levels at the end of this 3 weeks “patch-off” period. Symptoms, including the OSDI questionnaire were also recorded at all follow-up visits. Baseline blood results and OSDI questionnaire were collected from the patient's visit just before starting the Intrinsa® patch. Patients were followed up approximately 3 weeks later in the clinic. Thereafter the patients were followed up at approximately 6 weeks in the clinic after completion of cycles of 3 weeks patch-on and 3 weeks patch-off to review for any improvement in dry eye symptoms and to make a decision as to whether to continue therapy or not. During all the clinical visits, TFBUT and Schirmer's tests were performed on all patients. However, the OSDI questionnaires were either filled in by the patient at the time of clinical visits or when they did not have spare time at the clinical visits, they were filled over the phone later, or were sent out to them by post when the patient's were not available for this over the phone.

All data were entered in a Microsoft Excel sheet (Microsoft Office for Mac 2008, USA). Data on any coexisting ocular conditions, complications and side effects were also entered. A paired t test was used to compare the pre and post-patch TFBUT, Schirmer's test, serum testosterone, SHBG and OSDI scores. P<0.05 was considered significant.

### Results

Fourteen female patients (mean age 59.5±9.8 years, range 41-79 years, 13 Caucasian and 1 Indian) with meibomian gland dysfunction and EDE requested INTRINSA® patch therapy. Results of serum antinuclear antibodies, anti-ro and anti-la and serum rheumatoid factor were negative for all. Mean baseline serum testosterone was 0.64±0.69 nmol/l (range: 0-2.8 nmol/l) and serum SHBG was 85.02±49.17 nmol/l (range: 24-181 nmol/l). Mean baseline serum oestradiol levels were 172.1±197.0 nmol/l (range: 17-675 nmol/l) and serum progesterone levels were 1.4±1.3 nmol/l (range: 0.2-3.7 nmol/l). Before receiving androgen patch-therapy, 6 (43%) patients had undergone bilateral lower punctal occlusion with plugs.

All patients completed baseline OSDI questionnaires before referral for androgen patches. Eleven (78.5%) patients completed OSDI questionnaires immediately after 3 weeks patch-on, and 7 (50%) completed OSDI questionnaires immediately after 3 weeks patch off and subsequent 3 weeks patch on. Mean follow-up was 12.9±2.7 (range 9-18) months.

Mean BCVA (6/6) did not change post patching. In all 14 patients, the mean TFBUT in both eyes (pre-patch 2.96±1.12 sec Vs post-patch 6.7±2.2 sec, p<0.03) and Schirmer's test (pre-patch 6.0±2.9 sec Vs post-patch 8.8±2.0 sec, p<0.03) improved significantly after 3 weeks of androgen patching. Serum SHBG reports were available for 11 patients pre-patch and 6 patients post-patch. Serum testosterone levels were available for all patient pre-patching and 12 patients post-patching. Two patients who discontinued patch-therapy after 2 cycles but remained under follow-up did not request further patch treatment or any further blood tests. Changes in serum SHBG (pre-patch 85.02±49.17 nmol/l Vs post-patch 62.3±52.3 nmol/l, p=0.76) and serum testosterone (pre-patch 0.64±0.69 nmol/l Vs post-patch 1.56±1.0 nmol/l, p=0.07) did not reach statistical significance.

From the available OSDI questionnaires, there was a consistent and significant improvement in the symptom of ‘painful or sore eyes’ after patch-therapy (p<0.05) (FIGS. 1 and 2) and in ‘windy conditions’ (p=0.04) (FIG. 1). At the first cycle, there was no significant difference between mean total OSDI scores pre-patching (42±22.4) Vs 3 weeks patch-off (51.8±24.8) (p=0.33), whereas mean total OSDI scores were significantly different pre-patching (42±22.4) Vs 3 weeks patch on (13±27.6) (p<0.01). Moreover, mean total OSDI scores were significantly less after subsequent 3 weeks patch on (25.3±33.7) Vs 3 weeks patch-off (51.8±24.8) (p<0.01). Although the change in testosterone levels with use of the patch did not reach significance, the trend was an increase in serum testosterone. As the SHBG levels did not change or reduced, this would mean a relative increase in the level of free and active testosterone.

All patients reported a subjective improvement after wearing the patch. Six patients continued the patch for one year. Of the remaining patients, 3 discontinued after 2 cycles with no further improvement and 5 after 3 cycles, reporting either an improvement in their symptoms with less use of adjunctive therapy or other reasons.

Each patient was asked individually about any other systemic adverse effects. One patient reported sleep disturbance at night. One developed a hypersensitivity reaction to the patch adhesive and she subsequently was prescribed TESTOGEL® (Bayer, Australia) as an alternative. One patient developed hirsuitism around 6 months, which resolved after wearing the patch on alternate weekly basis (i.e. one week on and one week off). No other significant side effects reported in product specification for INTRINSA® (Drug Ther Bull 47(3):30-4, 2009) were observed in this study.

To the knowledge of the present inventors, this is the first case series describing such use of Intrinsa® for EDE management. TFBUT, Schirmer's test and OSDI scores improved after cycles of patch therapy. Patients reported a symptomatic improvement with cycles of patch-therapy with continued use of adjunctive treatment.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for treating dry eye syndrome, comprising: systemically administering to a patient in need thereof an effective amount of an androgen.

2. The method of claim 1, wherein the androgen is administered transdermally or subcutaneously.

3. The method of claim 1, wherein the androgen is administered transdermally via an androgen patch or gel.

4. The method of claim 1, wherein the androgen is administered subcutaneously via an androgen depot or implant.

5. The method of claim 1, wherein the androgen is administered at a dose range of 100-1300 μg per day.

6. The method of claim 1, wherein the androgen is testosterone.

7. The method of claim 1, wherein the dry eye syndrome is evaporative dry eye syndrome.

8. The method of claim 1, wherein the dry eye syndrome is aqueous tear-deficient dry eye syndrome.

9. The method of claim 1, wherein the patient has a low serum androgen level.
